(19) United States
(12) Patent Application Publication
(10) Pub. No.: US 2009/0324742 A1
(43) Pub. Date: Dec. 31, 2009

(54) Peham dendrimers as excipients

(76) Inventors: Sonke Svenson, Midland, MI (US); Abhay Singh Chauhan, Mt. Pleasant, MI (US)

Correspondence Address: TECHNOLOGY LAW, PLLC, 3595 N. SUNSET WAY, SANFORD, MI, 48657, US

(21) Appl. No.: 12/310,748

(22) PCT Filed: Sep. 7, 2007
(86) PCT No.: PCT/US07/19601

§ 371 Date: Mar. 5, 2009

Related U.S. Application Data
(60) Provisional application No. 60/843,047, filed on Sep. 8, 2006.

(51) Int. Cl. A61K 47/48; A61K 47/34; A61K 33/24; A61K 31/59; A61K 8/84

(52) U.S. Cl. 424/649; 514/772.3; 514/167

(57) ABSTRACT

The present invention concerns poly(etherhydroxylamine) PEHAM dendritic polymers wherein they function as excipients for the enhancement of water solubility of poorly water soluble (hydrophobic) Active Materials or enhancement of oil solubility of poorly oil soluble (hydrophilic) Active Materials. These dendritic polymers can have Active Materials associated with them by one or more of the following: (a) by adsorption onto the surface or (b) encapsulation into the interior of the dendritic polymers or (c) a mixture of both where these interactions are driven by one or more of the following (i) electrostatic attraction, (ii) hydrogen bonding between dendritic polymers and Active Material and (iii) hydrophobic or hydrophilic interactions or mixtures of these interactions. Additionally, these associated Active Materials can be associated with dendritic polymers through chemical bonding to the surface or to internal functionalities (IF) of PEHAM dendritic polymers or both. Such bonding is done either directly between PEHAM dendritic polymers and Active Material molecules or via a linker that can have a hydrolysable bond to the Active Material. In addition, a chemical entity with strong interaction to the Active Material and dendritic polymers can be associated with the dendritic polymer prior to adsorption or encapsulation of the Active Material or together with the Active Material.

### BACKGROUND OF THE INVENTION

1. Field of the Invention

The present invention concerns the field of dendritic polymers where poly(etherhydroxylamine) dendritic polymers (i.e., PEHAM dendritic polymers) and dendrimers are an example of the preferred polymers. These polymers have interior void spaces that may entrap molecules and their surface functionalities may undergo further reactions. By modification of the dendrimer structure and surface, PEHAM dendritic polymers can be utilized to enhance the solubility of poorly soluble active materials and change the properties of those active materials. These active materials can find applications in life science and medicine using pharmaceutical active ingredients (API) and diagnostic agents, and in cosmetics and in nutrition.

2. Description of Related Art

The use of highly water soluble excipients, such as polymers [i.e., poly(vinyl alcohol) and poly(ethylene oxide)-co-poly(propylene oxide) copolymers] or surfactants (i.e., the Brij® and Tween® surfactants) to enhance the solubility of poorly water soluble active pharmaceutical ingredients (API) such as drugs is well known in the preparation of pharmaceutical formulations. (See for example Fiedler Encyclopedia of Excipients for Pharmaceuticals, Cosmetics and Related Areas, pub. H. P. Fiedler, 2002.) The use of dendritic polymers, mainly poly(amidoamine) dendrimers (i.e., PAMAM dendrimers) to enhance the solubility of poorly soluble drugs has been studied as well [For example, S. Svenson, D. A. Tomalia, Advanced Drug Delivery Reviews 57, 2106 (2005)].

In one study, the use of PAMAM dendrimers G=4 with amino surface and G=4.5 with sodium carboxylate surface were studied on their ability to enhance the solubility of the drug indomethacin. [See A. S. Chauhan et al., J. Drug Targeting 12, 575, (2004).]

In another study, the encapsulation of the drug methotrexate into PAMAM dendrimers with various degrees of poly(ethylene glycol), molecular weight 2000, has been disclosed. [See G. Pan, Y. Lemmouchi, E. O. Akala, O. Bakare, J. Bioactive and Compatible Polymers 20, 113 (2005).] The chemical linkage of the drug methotrexate to PAMAM dendrimers having various surface functionalities and release characteristics has been disclosed by S. Gurdag et al., Bioconjugate Chemistry 17, 275 (2006).

Also poly(propyleneimine) dendrimers G=14 were studied for DNA delivery. [See A. G. Schatzlein et al., J. Controlled Release 101, 247 (2005).]

In another study, the use of PAMAM dendrimers G=1-5 with an amine surface for the delivery of drugs (e.g., ketoprofen, ibuprofen, diflunisal and naproxen) was reported by C. Yiyun et al., Europ. J. Med. Chem. 40, 1188 (2005).

In another study, the use of the drug methotraxate conjugated to PAMAM dendrimers G=5 with an amine surface was disclosed. [See T. P. Thomas et al., J. Med. Chem. 48, 3729 (2005).

In yet another study, the use of hyperbranched dendritic polyester (Boltomm H40) to carry the dye Congo Red as a drug model was disclosed. [See J. Zou et al., J. Phys. Chem. B 110, 2638 (2006).]

The effect of PAMAM dendrimers G=4 as a solubility enhancer for ibuprofen has been studied by O. M. Milhem et al., Int. J. Pharmaceutics 197, 239 (2000).

The effect of PAMAM dendrimers G=0-3 with either an amine or ester surface on the solubility of nifedipine has been disclosed by B. Devarakonda et al., Int. J. Pharmaceutics 284, 133 (2004).

These above referenced examples selectively describe the breadth of related dendrimer art but are not intended to provide an exhaustive overview over the use of dendrimers in enhanced drug solubility. The focus of the above related art is clearly on drug adsorption (a) onto dendrimer surfaces, or (b) physical encapsulation into dendrimer cores, or (c) chemical linkage onto the dendrimer surface.

Taking into consideration the cost and poor commercial scalability of PAMAM dendrimers, it clearly would be desirable to utilize other precise dendrimer structures made with a faster reaction time, easier separation with fewer by-products, and lower cost of manufacture than that presently available. Additionally, if the dendrimers were more stable and easier to scale, that also would be desirable to enhance product stability, extend product shelf lifetime, and allow for larger processing temperature ranges. These improved properties would widen the applicability of dendritic polymers into areas outside the life sciences and medical fields, such as cosmetics and nutrients.

### BRIEF SUMMARY OF THE INVENTION

None of the dendrimers described above carry internal functionalities (IF), a feature of PEHAM dendritic polymers, that would allow internal linkage of Active Material molecules or the use of chemically linked co-excipients as disclosed in this invention.

The PEHAM dendrimers of this invention have a precise dendrimer structure made with a faster reaction time, easier separation with fewer by-products, and lower cost of manufacture that provides better commercial scalability than that presently available for other dendrimers. Additionally, these dendrimers are more stable and easier to scale, with enhanced product stability, extended product shelf lifetime, and allows for a larger processing temperature range.

Particularly, poly(etherhydroxylamine) dendritic polymers (i.e., PEHAM dendritic polymers) are disclosed wherein they function as excipients for the enhancement of water solubility of poorly water soluble (hydrophobic) Active Materials or enhancement of oil solubility of poorly oil soluble (hydrophilic) Active Materials. These dendritic polymers can have Active Materials associated with them by one or more of the following: (a) by adsorption onto the surface, (b) encapsulation into the interior of the dendritic polymers, with or without covalent bonding or other linkage to the (IF) group, or (c) a mixture of both (a) and (b) where these interactions are driven by one or more of the following: (i) electrostatic attraction, (ii) hydrogen bonding between dendritic polymers and the Active Material and/or (iii) hydrophobic or hydrophilic interactions or mixtures of these interactions. Additionally, these associated Active Materials can be associated with dendritic polymers through chemical bonding to the surface or to internal functionalities (IF) of PEHAM dendritic polymers or both. Such bonding is done either directly between PEHAM dendritic polymers and Active Material molecules or via a linker that can have a hydrolysable bond to the Active Material. For example, an Active Material “Q” can be bound through appropriate chemical reaction only or mainly to the outside and an Active Material “X” bound only or mainly to the interior of the dendritic polymers, thereby creating a combination of Active Materials in one dendrimer. In the life sciences and medical fields, for example, these Active Materials would be drugs or diagnostic agents (e.g., pharmaceutical agents), thus allowing the preparation of combination therapy or drug cocktail. A chemical entity with strong interaction to the Active Material and dendritic polymer can be associated with the dendritic polymer through physical means prior to drug adsorption or encapsulation. Also the chemical entity can be administered together with the Active Material. Alternatively, the chemical entity can be chemically attached to (IF) or (TF) prior to association with the Active Material. Formulations of said dendritic polymers can be prepared by: (a) mixing a solid Active Material with pre-dissolved PEHAM dendritic polymer, (b) mixing a solid PEHAM dendritic polymer with pre-dissolved Active Material, (c) mixing both Active Material and dendrimer as solids and then dissolving both at the same time, or (d) mixing pre-dissolved Active Material and PEHAM dendritic polymer, wherein in the final mixture the dendritic polymer is more strongly associated with the Active Material than the bulk solution is associated with the Active Material so that there is a driving force for the Active Material to interact with the dendrimer.

### BRIEF DESCRIPTION OF THE DRAWINGS

FIG. 1 shows the solubility enhancement of indomethacin in the presence of PEHAM dendritic polymers G=1.5 with PETGE core and piperazine (NH) surface and G=2.0 with PETGE core and hydroxyl (OH) surface. The solubility enhancement factor x is shown as well.

FIG. 2 shows the solubility enhancement of the cosmetic and nutrient Active Material, vitamin D3 in the presence of PEHAM dendritic polymers G=1.0 and G=2.0 with PETGE (columns 4-7 from left) or TMPTGE (columns 1-3 from left) cores and amine surfaces, partially PEGylated (25% PEG-550) amine surfaces (denoted with letter “P”), or carboxylate surfaces. All but one of these PEHAM dendritic polymers increase the solubility of vitamin D3. The pure Active Material as control remains below detection limit of 50 ng/mL.

FIG. 3 shows the effect of PEHAM dendritic polymers on the dissolution rate of vitamin D3 in PBS solution. The otherwise poorly water soluble Active Material dissolves completely within 5 min. in the presence of PEHAM excipients.

FIG. 4 shows the solubility enhancement of indomethacin in the presence of PEHAM dendritic polymer G=1.5 with PETGE core and poly(ethylene glycol), molecular weight 550, surface in the presence (PEG-E) and absence (PEG) of co-excipient sodium 4-nitrophenylformiate. The solubility enhancement factor x is shown as well.

FIG. 5 shows the solubility enhancement of indomethacin in the presence of PEHAM dendritic polymers G=1.0 with PETGE core and piperazine (NH) surface and a PAMAM dendritic polymer G=4.0 with EDA core and amine surface. The solubility enhancement factor x is shown as well.

FIG. 6 shows the altered pharmacological release profiles of indomethacin associated with PEHAM dendritic polymers G=1.5 with PETGE core and piperazine (NH) or poly(ethylene glycol), molecular weight 550, (PEG-E) surface, G=2.0 with PETGE core and hydroxyl (OH) surface, and G=2.5 with TPEGE core and sodium carboxylate (COONa) surface in PBS solution at pH 7.25 and 37° C.

### DETAILED DESCRIPTION OF THE INVENTION

### Glossary

The following terms as used in this application are to be defined as stated below and for these terms, the singular includes the plural.
Active Material means any entity that has an influence on the body such as a pharmaceutical agent, e.g., a drug, a diagnostic agent, or a therapeutic agent, and any other active material used in cosmetic and nutrient formulations, whose function, e.g., solubility, will be improved for the desired application by association with a dendritic polymer
AEEA means N-(2-hydroxyethyl)ethylenediamine
amu means atomic mass units
API means active pharmaceutical ingredients, e.g., Active Material ingredients
Aptamer means a specific synthetic DNA or RNA oligonucleotide that can bind to a particular target molecule, such as a protein or metabolite
Associated with means that the carried Active Material(s), (M), can be physically encapsulated or entrapped within the interior of the dendrimer, dispersed partially or fully throughout the dendrimer, or attached or linked to the dendrimer or any combination thereof, whereby the attachment or linkage is by means of covalent bonding, hydrogen bonding, adsorption, absorption, metallic bonding, van der Walls forces or ionic bonding, or any combination thereof
BAA means bis(allyl)amine or diallylamine
DETA means diethylenetriamine
DI water means deionized water
DNA or RNA or nucleic acids means synthetic or natural, single or double stranded DNA or RNA or PNA (phosphorous nucleic acid) or combinations thereof or Aptamers, preferably from 4 to 9000 base pairs or from 500 D to 150 kD
EDA means ethylenediamine; Aldrich
Excipient means a material that interacts with the pharmaceutical or cosmetical or nutritional Active Material and enhances its solubility in the desired solvent
G means dendrimer generation, which is indicated by the number of concentric branch cell shells surrounding the core (usually counted sequentially from the core)
IDAN means 3,3-iminodiacetonitrile
(IF) means internal functionalities present in PEHAM dendrimers; e.g. hydroxyl, amines, thiols, or other groups capable of chemical bonding when from with the PEHAM dendrimer
IMAE means 2-imidazolidyl-1-aminoethane
IMPA means imino bis(methylphosphonic acid)
IR means infrared spectroscopy
MeOH means methanol
mg means milligram(s)
MIA means 2-methyl-2-imidazoline
mins. means minutes
μg means microgram(s)
mL means milliliter(s)
μm means micrometer(s)
ng means nanogram(s)
nm means nanometer(s)
Oligonucleotides means synthetic or natural, single or double stranded DNA or RNA or PNA (peptide nucleic acid) or combinations thereof or aptamers, preferably from 4 to 100 base pairs
PAMAM means poly(amidoamine), including linear and branched polymers or dendrimers with primary amine terminal groups
PBS means phosphate buffered saline
PEA means methyl isobutyl protected 1-(2-aminoethyl)piperazine
PEG means polyethylene glycol molecules of certain molecular weights (e.g., 550 Da), which are chemically bonded to the surface of dendritic polymers
PEG-E means polyethylene glycol molecules that are chemically or physically associated with a chemical entity that acts as an excipient, i.e., modifies the behavior of PEG
PEHAM means poly(etherhydroxylamine); dendrimers of Formula (I) below and as described in WO/2006/065266 and WO/2006/115547, which are incorporated herein by reference as to those structures and process to make them
PEI means poly(ethyleneimine)
Percent or % means by weight unless stated otherwise
PETAZ means pentaerythritol tetraazide
PETGE means pentaerythritol tetraglycidyl ether
PETriGE means pentaerythritol triglycidyl ether
PIPZ means piperazine or diethylenediamine
rpm means rotation per minute, the frequency of agitation in a shaking water bath
RT means ambient temperature or room temperature, about 20-25° C.
(TF) means a terminal functionality on or near the surface of a dendrimer
TMPTGE means trimethylolpropane triglycidyl ether
TMS means tetramethylsilane
TPEGE means tetraphenylolethane glycidyl ether
TPMTGE means triphenylolmethane triglycidyl ether
TREN means tris(2-aminoethyl)amine
UV-vis means ultraviolet and visible spectroscopy

Poly(etherhydroxylamine) dendritic polymers, as described in WO/2006/065266 and WO/2006/115547, and the process to make such dendrimers are described in these published references, which are hereby incorporated by reference. These PEHAM dendritic polymers are utilized in the present invention as excipients for the enhancement of water solubility of poorly water soluble (hydrophobic) Active Materials or enhancement of oil solubility of poorly oil soluble (hydrophilic) Active Materials. Active Materials can be associated with dendrimers by: (a) adsorption onto the surface, (b) encapsulation into the dendrimer interior, or (c) a mixture of both (a) and (b). The (TF) and (IF) groups of the PEHAM dendrimers are used to provide this functionality. These interactions are driven by one or more of various forces such as, but not limited to, electrostatic attraction, hydrogen bonding between dendrimer and Active Material, and hydrophobic or hydrophilic interactions or mixtures of these interactions.

Active Materials can be associated with dendrimers through chemical bounding to the surface (TF) or (IF) groups of PEHAM dendritic polymers or both. This bonding can be done directly between PEHAM dendrimers and Active Material molecules or via a linker that can have a hydrolysable bond to the Active Material, i.e., acid or base or enzyme or temperature or light labile (e.g., IR light, which can penetrate tissue). Bonding of an Active Material can involve all functionalities available on PEHAM surface/interior or only a fraction of these functionalities.

Through appropriate chemical reaction it is possible to chemically bind an Active Material “Q” only or mainly to the outside and an Active Material “X” only or mainly to the interior, this way creating a combination of Active Materials. In the life sciences and medical fields, for example, these Active Materials would be drugs, thus allowing the preparation of combination therapy or drug cocktail or could allow for a drug and a diagnostic to both be present with the PEHAM dendritic polymer.

A chemical entity with strong interaction to the Active Material and dendrimer can be associated with the dendrimer through physical means prior to adsorption or encapsulation of the Active Material or together with the Active Material. The entity will act as a co-excipient or co-encapsulant and enhance adsorption or encapsulation efficiency of the Active Material. A chemical entity with strong interaction to the Active Material and dendrimer can be chemically attached to (IF) or (TF) prior to adsorption or encapsulation of the Active Material. The presence of said entity will enhance the Active Materials' adsorption and encapsulation efficiency.

To prepare the formulation, any of the following methods can be used: (a) solid Active Material can be mixed with pre-dissolved PEHAM dendritic polymer, (b) solid PEHAM dendritic polymer can be mixed with pre-dissolved Active Material, (c) both Active Material and dendrimer can be mixed as solids and then dissolved at the same time, or (d) both Active Material and PEHAM dendritic polymer can be pre-dissolved and then mixed as solutions. However, it is important that in the final mixture the dendritic polymer is more strongly associated with the Active Material than the bulk solution is associated with the Active Material so that there is a driving force for the Active Material to interact with the dendrimer.

Loading efficiency of Active Material materials into PEHAM dendritic polymers is higher than those observed for other dendrimers and is achieved at a lower generation (so less time to make them is involved). Additionally, the high thermal stability of PEHAM dendritic polymers allows thermal sterilization of pharmaceutical formulations, which was not possible with PAMAM dendritic polymers.

PEHAM-Active Materials formulations can be stored and provided as a powder mixture and re-dissolved prior to application. PEHAM-Active Materials formulations can be prepared as a solid mixture and pressed into tablets. PEHAM-Active Materials formulations can be prepared by concentration of mixed solutions and stored and provided as a suspension or paste filled into a capsule.

PEHAM-Active Materials formulations can be administered by an oral route, ampoule, intravenous injection, intramuscular injection, transdermal application, intranasal application, intraperitoneal administration, subcutaneous injection, ocular application, as wipes, sprays, gauze or other means for use at a surgical incision, near scar formation sites, or site of a tumor growth or removal or near or within a tumor.

PEHAM-Active Materials formulations can provide a more desirable pharmacological profile of the respective active in the case of the Active Material being a drug or diagnostic agent or can improve the interaction with the body and desired performance in case of cosmetics and nutrients.

### Chemical Structure

The PEHAM dendritic polymer structures of the present invention possess several unique components that manifest surprising properties compared to traditional dendritic structures and utilize unique ring opening processes for their preparation.

A structure for these dendritic polymers is shown by Formula (I) below and described in WO/2006/065266 (i.e., pp. 6-7; pp. 13-22; pp. 23-38) and WO/2006/115547 (i.e., pp. 6-8; pp. 23-34), which is hereby incorporated by reference.

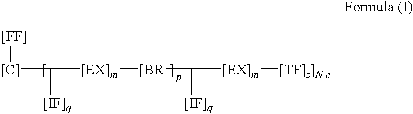

wherein:
(C) means a core;
(FF) means a focal point functionality component of the core;
x is independently 0 or an integer from 1 to N_{c}−1;
(BR) means a branch cell, which, if p is greater than 1, then (BR) may be the same or a different moiety;
p is the total number of branch cells (BR) in the dendrimer and is an integer from 1 to 2000 derived by the following equation

$$p = Total \# of [ BR ] \\ = ( \frac{{N}_{b}^{1}}{{N}_{b}} + \frac{{N}_{b}^{2}}{{N}_{b}} + \frac{{N}_{b}^{3}}{{N}_{b}} + … \frac{{N}_{b}^{G}}{{N}_{b}} ) [ {N}_{c} ] \\ = ( {\sum }_{i = 0}^{i = G - 1} {N}_{b}^{i} ) [ {N}_{c} ]$$

where: G is number of concentric branch cell shells (generation) surrounding the core;
   i is final generation G;
   N_{b} is branch cell multiplicity; and
   N_{c} is core multiplicity and is an integer from 1 to 1000;
(IF) means interior functionality, which, if q is greater than 1, then (IF) may be the same or a different moiety;
q is independently 0 or an integer from 1 to 4000;
(EX) means an extender, which, if m is greater than 1, then (EX) may be the same or a different moiety;
m is independently 0 or an integer from 1 to 2000;
when both q and m are greater than 1, (BR) and (EX) may occur alternately with the other moiety or sequentially with multiple groups of (BR) or (EX) occurring in succession;
(TF) means a terminal functionality, which, if z is greater than 1, then (TF) may be the same or a different moiety;
z means the number of surface groups from 1 to the theoretical number possible for (C) and (BR) for a given generation G and is derived by the following equation

z=N_{c}N_{b}^{G};
where: G, N_{b} and N_{c} are defined as above; and

with the proviso that at least one of (EX) or (IF) is present.

Preferred compounds of Formula (I) above are those where N_{c} is an integer from 1 to 20; q is 0 or an integer from 1 to 250 at each occurrence; p is an integer from 1 to 250 at each occurrence; and m is 0 or an integer from 1 to 250 at each occurrence; and one of q or m must be at least 1; and when both q and m are greater then 1, (BR) and (EX) may occur alternately with the other moiety or sequentially with multiple groups of (BR) or (EX) occurring in succession. Thus each generation can have a different sequence of these (BR) and (EX). Preferably the (IF) is present in the compound of Formula (I).

Other preferred dendritic polymers of Formula (I) are those where one or more of the following moieties are present: where (C) is PETriGE, PETAZ, TPEGE, or TPMTGE; or where (BR) is IDAN, IMAE, IMPA, BAA, DETA, TREN, AEEA, or MIA; or where (TF) is TMS; or where (EX) is PIPZ or triazole.

In the above Formula (I) the terms used are further explained in the published PCT applications WO/2006/065266 and WO/2006/115547.

Thus prepared, the dendrimer of Formula (I) can be reacted with a wide variety of compounds to produce polyfunctional compounds with unique characteristics as disclosed in WO/2006/065266 (pp. 20-22) and WO/2006/115547 (pp. 34-58).

### Dendritic Excipient:

An excipient in this invention is defined as a dendritic polymer that interacts with the pharmaceutical or cosmetical or nutritional Active Material and enhances its solubility in the desired solvent. In addition, the presence of the excipient might alter the pharmacological profile of the respective Active Material, reduce its toxicity or its retention time within the body or uptake by the body or general interaction with the body, although these activities are not its main purpose. The PEHAM dendritic polymer acting as an excipient might by itself be inactive or active in the respective application; however it must exert a solubility enhancing effect upon the Active Material it is formulated with to be a part of this invention.

Active Material Associated with PEHAM Dendritic Polymers:

The association of the carried Active Material(s) and the dendrimer(s) may optionally employ connectors and/or spacers or chelating agents to facilitate the preparation or use of these conjugates. Suitable connecting groups are groups which link a targeting director (i.e., T) to the dendrimer (i.e., D) without significantly impairing the effectiveness of the director or the effectiveness of any other carried Active Material(s) (i.e., M) present in the combined dendrimer and material (“conjugate”). These connecting groups may be cleavable or non-cleavable and are typically used in order to avoid steric hindrance between the target director and the dendrimer, preferably the connecting groups are stable (i.e., non-cleavable) unless the site of delivery would have a cleavable linker present (e.g., an acid-cleavable linker at the cell surface). Since the size, shape and functional group density of these dendrimers can be rigorously controlled, there are many ways in which the carried material can be associated with the dendrimer. For example, (a) there can be covalent, coulombic, hydrophobic, or chelation type association between the carried material(s) and entities, typically functional groups, located at or near the surface of the dendrimer; (b) there can be covalent, coulombic, hydrophobic, or chelation type association between the carried material(s) and moieties located within the interior of the dendrimer; (c) the dendrimer can be prepared to have an interior which is predominantly hollow allowing for physical entrapment of the carried materials within the interior (void volume), wherein the release of the carried material can optionally be controlled by congesting the surface of the dendrimer with diffusion controlling moieties, (d) where the dendrimer has internal functionality groups (IF) present which can also associate with the carrier material, or (e) various combinations of the aforementioned phenomena can be employed.

The Active Material (M) that is encapsulated or associated with these dendrimers may be a very large group of possible moieties that meet the desired purpose. Such materials include, but are not limited to, pharmaceutical materials for in vivo or in vitro or ex vivo use as diagnostic or therapeutic treatment of animals or plants or microorganisms, viruses and any living system, which material can be associated with these dendrimers without appreciably disturbing the physical integrity of the dendrimer. Examples of (M) are given in WO/2006/115547 (i.e., pp. 61-65), which is hereby incorporated by reference.

### Methods of Making the PEHAM Dendritic Polymers of Formula (I):

The current invention involves PEHAM dendritic polymers that have been built using branch cell reagents, which are typically bulky, multifunctional molecules compared to the smaller reagents (i.e., ethylenediamine and methyl acrylate) described in typical divergent PAMAM synthesis processes. Details of the methods of making have been disclosed in WO/2006/065266 (i.e., pp. 23-26) and WO/2006/115547 (i.e., pp. 37-58), which is hereby incorporated by reference.

In summary, the making of PEHAM dendritic polymers involves the use of faster, kinetically driven, reactive ring-opening chemistry (i.e., “click type” or other fast reactions) combined with the use of more bulky, polyfunctional branch cell reagents (BR) in a controlled way to rapidly and precisely build dendrimer structures, generation by generation. This process provides precise structures with cleaner chemistry, typically single products, requires lower excesses of reagents, lower levels of dilution, thus offering a higher capacity method which is more easily scaled to commercial dimensions, new ranges of materials, and lower cost. The dendrimer compositions prepared possess novel internal functionality, greater stability, e.g., thermal stability and exhibit less or no reverse Michael's reaction (compared with traditional PAMAM dendrimer structures). Furthermore, they reach encapsulation surface densities (i.e., acquire nano-container properties) at lower generations (and therefore at less cost) than traditional PAMAM dendrimer structures. Unexpectedly, these present reactions of poly-functional branch cell reagents (BR), possessing highly functionalized surfaces do not lead to gelled, bridged/cross-linked systems/materials even at lower stoiochiometries/excesses than normally required for traditional PAMAM dendrimer systems.

Divergent dendritic growth can be precisely controlled to form ideal dendritic polymers which obey mathematical formulas, at least through the first several generations of growth. However, because the radii of dendrimer molecules increase in a linear manner as a function of generation during ideal divergent growth, whereas the surface cells amplify according to a geometric progression law, ideal dendritic growth does not extend indefinitely. There is a critical generation at which the reacting dendrimer surface does not have enough space to accommodate incorporation of all of the mathematically required new units. This stage in digression from ideal dendritic growth is referred to as the de Gennes dense-packed stage. At this stage, the surface becomes so crowded with terminal functional groups that, although the terminal groups are chemically reactive, they are sterically prohibited from participating further in ideal dendritic growth. In other words, the de Gennes dense-packed stage is reached in divergent dendrimer synthesis when the average free volume available to the reactive terminal group decreases below the molecular volume required for the transition state of the desired reaction to extend the dendritic growth to the next generation. Nevertheless, the appearance of the de Gennes dense-packed stage in divergent synthesis does not preclude further dendritic growth beyond this point. It has been demonstrated by mass spectrographic studies that further increase in the molecular weight can occur beyond the de Gennes dense-packed stage. However, this occurs in a non-ideal fashion that no longer adheres to values predicted by dendritic mathematics.

Products resulting from continuation of dendritic growth beyond the dense-packed stage are “imperfect” in structure, because some of the surface groups in the precursor generation are sterically precluded from undergoing further reaction. The number of functional groups on a dendrimer which has been grown past the de Gennes dense-packed stage will not correspond to the ideal, mathematically predicted value for that generation. This discontinuity is interpreted as a signature for the de Gennes dense-packed stage.

Definition of “Active Material (M)” Associated with PEHAM Dendritic Polymer:

The material (M) that is encapsulated or associated with these dendrimers may be a very large group of possible moieties that meet the desired purpose. Such materials include, but are not limited to, pharmaceutical materials for in vivo or in vitro or ex vivo use as diagnostic or therapeutic treatment of animals or plants or microorganisms, viruses and any living system, which material can be associated with these dendrimers without appreciably disturbing the physical integrity of the dendrimer.

In a preferred embodiment, the carried materials, herein represented by “M”, are pharmaceutical materials. Such materials which are suitable for use in the present dendrimer conjugates include any materials for in vivo or in vitro use for diagnostic or therapeutic treatment of mammals which can be associated with the dendrimer without appreciably disturbing the physical integrity of the dendrimer, for example: drugs, such as antibiotics, analgesics, hypertensives, cardiotonics, steroids and the like, such as acetaminophen, acyclovir, alkeran, amikacin, ampicillin, aspirin, bisantrene, bleomycin, neocardiostatin, chloroambucil, chloramphenicol, cytarabine, daunomycin, doxorubicin, cisplatin, carboplatin, fluorouracil, taxol, gemcitabine, gentamycin, ibuprofen, kanamycin, meprobamate, methotrexate, novantrone, nystatin, oncovin, phenobarbital, polymyxin, probucol, procarbabizine, rifampin, streptomycin, spectinomycin, symmetrel, thioguanine, tobramycin, trimethoprim, and valbanl; toxins, such as diphtheria toxin, gelonin, exotoxin A, abrin, modeccin, ricin, or toxic fragments thereof; metal ions, such as the alkali and alkaline-earth metals; radionuclides, such as those generated from actinides or lanthanides or other similar transition elements or from other elements, such as ⁴⁷Sc, ⁶⁷Cu, ⁶⁷Ga, ⁸²Rb, ⁸⁹Sr, ⁸⁸Y, ⁹⁰Y, ^{99m}Tc, ¹⁰⁵Rh, ¹⁰⁹Pd, ¹¹¹In, ^{115m}In, ¹²⁵I, ¹³¹I, ¹⁴⁰Ba, ¹⁴⁰La, ¹⁴⁹Pm, ¹⁵³Sm, ¹⁵⁹Gd, ¹⁶⁶Ho, ¹⁷⁵Yb, ¹⁷⁷Lu, ¹⁸⁶Re, ¹⁸⁸Re, ¹⁹⁴Ir, and ¹⁹⁹Au, preferably ⁸⁸Y, ⁹⁰Y, ^{99m}Tc, ¹²⁵I, ¹³¹I, ¹⁵³Sm, ¹⁶⁶Ho, ¹⁷⁷Lu, ¹⁸⁶Re, ⁶⁷Ga, ¹¹¹In, ¹¹⁵In, and ¹⁴⁰La; signal generators, which includes anything that results in a detectable and measurable perturbation of the system due to its presence, such as fluorescing entities, phosphorescence entities and radiation; signal reflectors, such as paramagnetic entities, for example, Fe, Gd, or Mn; chelated metal, such as any of the metals given above, whether or not they are radioactive, when associated with a chelant; signal absorbers, such as near infrared, contrast agents (such as imaging agents and MRI agents) and electron beam opacifiers, for example, Fe, Gd or Mn; antibodies, including monoclonal or polyclonal antibodies and anti-idiotype antibodies; antibody fragments; aptamers; hormones; biological response modifiers such as interleukins, interferons, viruses and viral fragments; diagnostic opacifiers; and fluorescent moieties. Carried pharmaceutical materials include scavenging agents such as chelants, antigens, antibodies, aptamers, or any moieties capable of selectively scavenging therapeutic or diagnostic agents.

In another embodiment, the carried materials, herein represented by “M”, are agricultural materials. Such materials which are suitable for use in these conjugates include any materials for in vivo or in vitro treatment, diagnosis, or application to plants or non-mammals (including microorganisms) which can be associated with the dendrimer without appreciably disturbing the physical integrity of the dendrimer. For example, the carried materials can be toxins, such as diphtheria toxin, gelonin, exotoxin A, abrin, modeccin, ricin, or toxic fragments thereof; metal ions, such as the alkali and alkaline earth metals; radionuclides, such as those generated from actinides or lanthanides or other similar transition elements or from other elements, such as ⁴⁷Sc, ⁶⁷Cu, ⁶⁷Ga, ⁸²Rb, ⁸⁹Sr, ⁸⁸Y, ⁹⁰Y, ^{99m}Tc, ¹⁰⁵Rh, ¹⁰⁹Pd, ¹¹¹In, ^{115m}In, ¹²⁵I, ¹³¹I, ¹⁴⁰Ba, ¹⁴⁰La, ¹⁴⁹Pm, ¹⁵³Sm, ¹⁵⁹Gd, ¹⁶⁶Ho, ¹⁷⁵Yb, ¹⁷⁷Lu, ¹⁸⁶Re, ¹⁸⁸Re, ¹⁹⁴Ir, and ¹⁹⁹Au; signal generators, which includes anything that results in a detectable and measurable perturbation of the system due to its presence, such as fluorescing entities, phosphorescence entities and radiation; signal reflectors, such as paramagnetic entities, for example, Fe, Gd, or Mn; signal absorbers, such contrast agents and as electron beam opacifiers, for example, Fe, Gd, or Mn; hormones; biological response modifiers, such as interleukins, interferons, viruses and viral fragments; pesticides, including antimicrobials, algaecides, arithelmetics, acaricides, H insecticides, attractants, repellants, herbicides and/or fungicides, such as acephate, acifluorfen, alachlor, atrazine, benomyl, bentazon, captan, carbofuran, chloropicrin, chlorpyrifos, chlorsulfuron cyanazine, cyhexatin, cypermithrin, 2,4-dichlorophenoxyacetic acid, dalapon, dicamba, diclofop methyl, diflubenzuron, dinoseb, endothall, ferbam, fluazifop, glyphosate, haloxyfop, malathion, naptalam; pendimethalin, permethrin, picloram, propachlor, propanil, sethoxydin, temephos, terbufos, trifluralin, triforine, zineb, and the like. Carried agricultural materials include scavenging agents such as chelants, chelated metal (whether or not they are radioactive) or any moieties capable of selectively scavenging therapeutic or diagnostic agents.

In another embodiment, the carried material, herein represented by (M), are immuno-potentiating agents. Such materials which are suitable for use in these conjugates include any antigen, hapten, organic moiety or organic or inorganic compounds which will raise an immuno-response which can be associated with the dendrimers without appreciably disturbing the physical integrity of the dendrimers. For example, the carried materials can be synthetic peptides used for production of vaccines against malaria (U.S. Pat. No. 4,735,799), cholera (U.S. Pat. No. 4,751,064) and urinary tract infections (U.S. Pat. No. 4,740,585), bacterial polysaccharides for producing antibacterial vaccines (U.S. Pat. No. 4,695,624) and viral proteins or viral particles for production of antiviral vaccines for the prevention of diseases such as AIDS and hepatitis.

The use of these conjugates as carriers for immuno-potentiating agents avoids the disadvantages of ambiguity in capacity and structure associated with conventionally known classical polymer architecture or synthetic polymer conjugates used to give a macromolecular structure to the adjuvant carrier. Use of these dendrimers as carriers for immuno-potentiating agents, allows for control of the size, shape and surface composition of the conjugate. These options allow optimization of antigen presentation to an organism, thus resulting in antibodies having greater selectivity and higher affinity than the use of conventional adjuvants. It may also be desirable to connect multiple antigenic peptides or groups to the dendrimer, such as attachment of both T- and B-cell epitopes. Such a design would lead to improved vaccines.

It may also be desirable to conjugate pesticides or pollutants capable of eliciting an immune response, such as those containing carbamate, triazine or organophosphate constituents, to a dendrimer. Antibodies produced to the desired pesticide or pollutant can be purified by standard procedures, immobilized on a suitable support and be used for subsequent detection of the pesticide or pollutant in the environment or in an organism.

In a further embodiment, the carried materials, herein represented by “M”, which are suitable for use in these conjugates include any materials other than agricultural or pharmaceutical materials which can be associated with these dendrimers without appreciably disturbing the physical integrity of the dendrimer, for example: metal ions, such as the alkali and alkaline-earth metals; signal generators, which includes anything that results in a detectable and measurable perturbation of the system due to its presence, such as fluorescing entities, phosphorescence entities, infrared, near infrared, and radiation; signal reflectors, such as paramagnetic entities, for example, Fe, Gd, or Mn; signal absorbers, such as contrast agents and an electron beam opacifiers, for example, Fe, Gd, or Mn; pheromone moieties; fragrance moieties; dye moieties; and the like. Carried materials include scavenging agents such as chelants or any moieties capable of selectively scavenging a variety of agents.

Preferably the carried materials (M) are bioactive agents. As used herein, “bioactive” refers to an active entity such as a molecule, atom, ion and/or other entity which is capable of detecting, identifying, inhibiting, treating, catalyzing, controlling, killing, enhancing or modifying a targeted entity such as a protein, glycoprotein, lipoprotein, lipid, a targeted disease site or targeted cell, a targeted organ, a targeted organism [for example, a microorganism, plant or animal (including mammals such as humans)] or other targeted moiety. Also included as bioactive agents are genetic materials (of any kind, whether oligonucleotides, fragments, or synthetic sequences) that have broad applicability in the fields of gene therapy, siRNA, diagnostics, analysis, modification, activation, anti-sense, silencing, diagnosis of traits and sequences, and the like. These conjugates include effecting cell transfection and bioavailability of genetic material comprising a complex of a dendritic polymer and genetic material and making this complex available to the cells to be transfected.

These conjugates may be used in a variety of in vivo, ex vivo or in vitro diagnostic or therapeutic applications. Some examples are the treatment of diseases such as cancer, autoimmune disease, genetic defects, central nervous system disorders, infectious diseases and cardiac disorders, diagnostic uses such as radioimmunossays, electron microscopy, PCR, enzyme linked immunoadsorbent assays, nuclear magnetic resonance spectroscopy, contrast imaging, immunoscintography, and delivering pesticides, such as herbicides, fungicides, repellants, attractants, antimicrobials or other toxins. Non-genetic materials are also included such as interleukins, interferons, tumor necrosis factor, granulocyte colony stimulating factor, and other protein or fragments of any of these, antiviral agents.

These conjugates may be formulated into a tablet using binders known to those skilled in the art. Such dosage forms are described in Remington's Pharmaceutical Sciences, 18^{th} ed. 1990, pub. Mack Publishing Company, Easton, Pa. Suitable tablets include compressed tablets, sugar-coated tablets, film-coated tablets, enteric-coated tablets, multiple compressed tablets, controlled-release tablets, and the like. Ampoules, ointments, gels, suspensions, emulsions, injections (intramuscular, intravenous, intraperitoneal) may also be used as a suitable formulation. Customary pharmaceutically-acceptable salts, adjuvants, diluents and excipients may be used in these formulations. For agricultural uses these conjugates may be formulated with the usual suitable vehicles and agriculturally acceptable carrier or diluent, such as emulsifiable concentrates, solutions, and suspensions.

### Preparation of Solubility Enhancing Formulations Containing PEHAM Dendritic Polymers

Poly(etherhydroxylamine) PEHAM dendritic polymers, as described in WO/2006/065266 and WO/2006/115547, can be utilized as excipients for the enhancement of water solubility of poorly water soluble (hydrophobic) Active Materials or enhancement of oil solubility of poorly oil soluble (hydrophilic) Active Materials. Active Materials can be associated with dendrimers by adsorption onto the surface or encapsulation into the dendrimer interior or a mixture of both. These interactions are driven by electrostatic attraction, hydrogen bonding between dendrimer and Active Material and hydrophobic or hydrophilic interactions or mixtures of these interactions, to name a few forces.

Active Materials can be associated with dendrimers through chemical bonding to the surface or internal functionalities (IF) of PEHAM dendritic polymers or both. This bonding can be done directly between PEHAM dendrimers and Active Material molecules or via a linker that can have a hydrolysable bond to the Active Material, i.e., acid or base or enzyme or temperature or light (e.g., IR light, which can penetrate tissue) labile. Bonding of Active Materials can cover all functionalities available on PEHAM surface/interior or only a fraction of these functionalities.

Through appropriate chemical reaction it is possible to chemically bind an Active Material A only or mainly to the outside and an Active Material B only or mainly to the interior, this way creating a combination of Active Materials. In the life sciences and medical fields, for example, these Active Materials would be drugs, thus allowing the preparation of combination therapy or drug cocktail or a diagnostic and therapeutic agent combined.

A chemical entity with strong interaction to the Active Material and dendrimer can be associated with the dendrimer through physical means prior to adsorption or encapsulation of the Active Material or together with the Active Material. The entity will act as a co-excipient or co-encapsulant and enhance the Active Material's adsorption or encapsulation efficiency. A chemical entity with strong interaction to the Active Material and dendrimer can be chemically attached to (IF) prior to adsorption or encapsulation of the Active Material. The presence of said entity will enhance the Active Material's adsorption and encapsulation efficiency.

To prepare the formulation, solid Active Material can be mixed with pre-dissolved PEHAM dendritic polymer or solid PEHAM dendritic polymer can be mixed with pre-dissolved Active Material or both Active Material and dendrimer can be mixed as solids and then dissolved at the same time or both Active Material and PEHAM dendritic polymer can be pre-dissolved and then mixed as solutions. However, it is important that in the final mixture the dendritic polymer is more strongly associated with the Active Material than the bulk solution is associated with the Active Material so that there is a driving force for the Active Material to interact with the dendrimer.

Loading efficiency of Active Materials into PEHAM dendritic polymers is higher than those observed for other dendrimers and is achieved at lower generation.

The high thermal stability of PEHAM dendritic polymers allows thermal sterilization of formulations incorporating the Active Material.

PEHAM-Active Material formulations can be stored and provided as a powder mixture and re-dissolved prior to application. PEHAM-Active Material formulations can be prepared as a solid mixture and pressed into tablets. PEHAM-Active Material formulations can be prepared by concentration of mixed solutions and stored and provided as a suspension or paste filled into a capsule.

PEHAM-Active Material formulations can be administered by an oral route, ampoule, intravenous injection, intramuscular injection, transdermal application, intranasal application, intraperitoneal administration, subcutaneous injection, ocular application, as wipes, sprays, gauze or other means for use at a surgical incision, near scar formation sites, or site of a tumor growth or removal or near or within a tumor.

PEHAM-Active Materials formulations can provide a more desirable pharmacological profile of the respective Active Material in the case of the Active Material being a drug or can improve the interaction with the body and desired performance in case of cosmetics and nutrients.

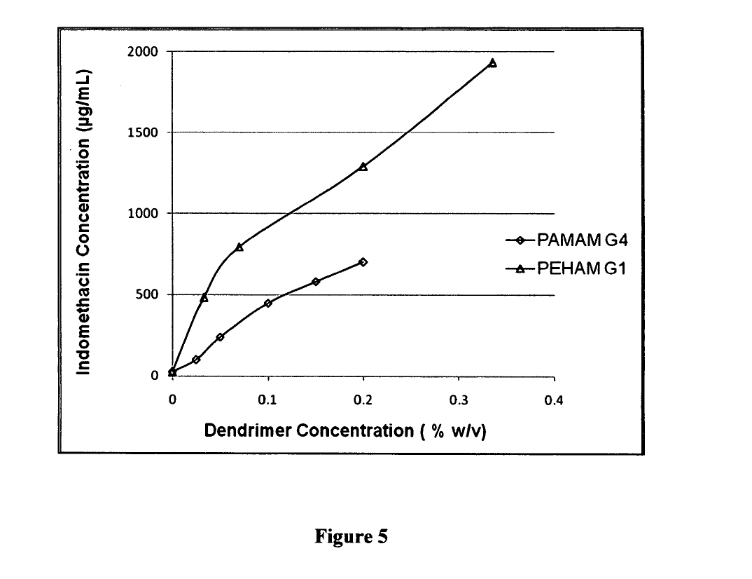

Some advantages of PEHAM dendritic polymers when compared to PAMAM dendritic polymer are that the PEHAM dendritic polymers are growing faster than PAMAM dendritic polymers, i.e., a generation G1 PEHAM has the size of a generation G2 PAMAM and similarly in later generation. As a consequence, PEHAM dendritic polymers generally have higher association efficiencies for Active Material than PAMAM by factors of about two to about forty in some cases, and by factors about two to about twenty in most cases. One example for higher association efficiency is disclosed in Example 7 and illustrated by FIG. 5.

For the following examples the various equipment and methods were used to run the various described tests for the results reported in the examples described below.

### Equipment and Methods

### High Pressure/Performance Liquid Chromatography (HPLC)

High pressure liquid chromatography (HPLC) was carried out using a Perkin Elmer™ Series 200 apparatus equipped with refractive index and ultraviolet light detectors and a Waters Symmetry® C₁₈ (5 μm) column (4.6 mm diameter, 150 mm length). A typical separation protocol was comprised of 0.1% aqueous acetic acid and acetonitrile (75:25% v/v) as the eluant and UV light at λ=480 nm as the detector.

### Dialysis Separation

In a typical dialysis experiment about 500 mg of product is dialyzed through a dialysis membrane with an appropriate pore size to retain the product and not the impurities. Dialyses are done in most examples in water (other appropriate dialyzates used were acetone and methanol) for about 21 hours with two changes of dialyzate. Water (or other dialyzate) is evaporated from the retentate on a rotary evaporator and the product dried under high vacuum or lyophilized to give a solid.

### Ultraviolet/Visible Spectrometry (UV/Vis)

UV-VIS spectral data were obtained on a Perkin Elmer™ Lambda 2 UV/VIS Spectrophotometer using a light wavelength with high absorption by the respective sample, for example 480 or 320 nm.

The invention will be further clarified by a consideration of the following examples, which are intended to be purely exemplary of the present invention.

### Example 1

### Solubility Enhancement of an Active Material, Indomethacin, Associated with PEHAM Dendritic Polymers G=1.5 with PETGE Core and PIPZ (NH) Surface and G=2.0 with PETGE Core and Hydroxyl (OH) Surface

[(C)=PETGE; (IF1)=OH; (EX1)=PIPZ; (IF2)=OH; (BR1)=PETGE; (IF3)=OH; (EX2)=PIPZ; (TF)=Secondary NH; G=1.5]
[(C)=PETGE; (IF1)=OH; (EX1)=PIPZ; (IF2)=OH; (BR1)=PETGE; (IF3)=OH; (EX2)=PIPZ; (BR2)=Glycidol; (TF)=OH; G=2.0]

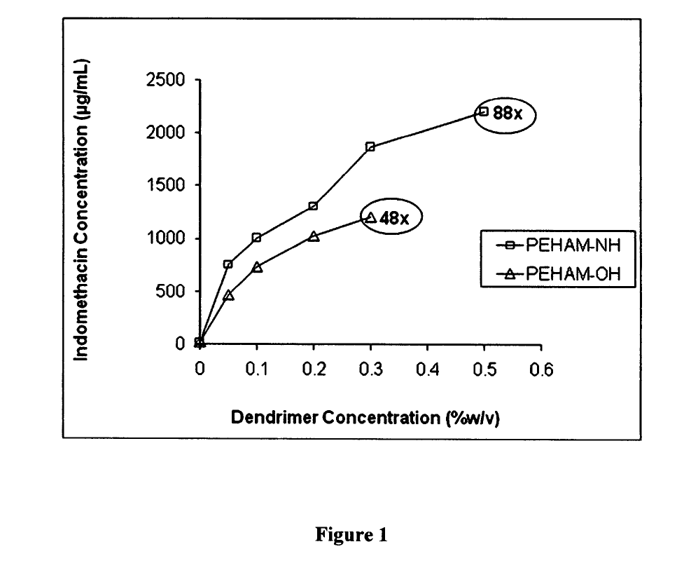

An excess of the Active Material, indomethacin (Alfa Aesar) was added to vials containing PEHAM dendritic polymers G=1.5 with PETGE core and PIPZ (NH) surface and G=2.0 with PETGE core and hydroxyl (OH) surface, each dissolved in DI water at concentrations of 0, 0.1, 0.2, 0.3, 0.4 and 0.5 percent (weight/volume). These suspensions were briefly ultrasonicated in a water bath at RT, then incubated overnight at 32° C. and 100 rpm in a shaking water bath, and allowed to equilibrate at RT. The suspensions were filtered through a 0.2-μm Nylon syringe filter to remove not encapsulated drug. Filtrates were analyzed for dendrimer-associated indomethacin by UV spectroscopy at 320 nm on a Perkin Elmer Lambda 2 UV/VIS Spectrometer. Association results are expressed as molar ratio indomethacin (I)/dendrimer (D). The results show enhanced indomethacin solubility for both PEHAM dendrimers with increasing PEHAM concentration. An increase by factor 88× at 0.5% w/v was observed for PEHAM-NH, and an increase by factor 48× at 0.3% w/v) was observed for PEHAM-OH (see FIG. 1). This FIG. 1 shows the solubility enhancement of indomethacin in the presence of PEHAM dendritic polymers G=1.5 with PETGE core and PIPZ (NH) surface and G=2.0 with PETGE core and hydroxyl (OH) surface. The solubility enhancement factor x is shown as well.

### Example 2

### Solubility Enhancement of an Active Material, Cisplatin, Associated with PEHAM Dendritic Polymer G=2.5 with TPEGE Core and Sodium Carboxylate (COONa) Surface

[(C)=TPEGE; (IF1)=OH; (BR1)=TREN; (EX1)=Methyl acrylate; (TF)=COONa; G=2.5]

PEHAM dendritic polymer G=2.5 with TPEGE core and sodium carboxylate surface (61.5 mg, 0.024 mM) was added to 60 mL DI water in a round bottom flask under shaking. Cisplatin (226 mg, 0.75 mM) was added to this solution, followed by ultrasonication for 5 mins. and heating at 50° C. for 20 mins. The reaction mixture was stirred at RT for 20 hours. Not encapsulated cisplatin was removed by dialysis (cut-off pore size 1000 Da) against 500 mL of DI water at 4° C. for 30 mins. Inside content of the dialysis bag was lyophilized and the platinum content measured by inductively coupled plasma spectroscopy (ICP). The platinum content was found to be 44.9±1.89% wt, (N=2).

### Example 3

### Solubility Enhancement of an Active Material, Paclitaxel, Associated with PEHAM Dendritic Polymer G=1.5 with PETGE Core and PIPZ (NH) Surface

[(C)=PETGE; (IF1)=OH; (EX1)=PEA; (TF)=Secondary NH; G=1.5]

Paclitaxel (1 mg) was added into a vial containing 2 mL of a 1% solution of PEHAM dendritic polymer G=1.5 with PETGE core and PIPZ (NH) surface in DI water. The sample was subjected to brief ultrasonication and heating at 50° C. for 10 mins., followed by overnight shaking at RT. The sample was then centrifuged for 5 mins. and the paclitaxel content measured by HPLC. The aqueous solubility of paclitaxel in the presence of PEHAM-NH was observed to be 8.13±0.24 μg/mL, compared to a solubility of 0.3 μg/mL paclitaxel in DI water without dendrimer.

### Example 4

### Solubility Enhancement of a Cosmetic and Nutrient Active Material, Vitamin D3, Associated with Various PEHAM Dendritic Polymers G=1.0 and G=2.0 with PETGE or TMPTGE Cores and Amine, Partially PEGylated Amine, or Carboxylate Surfaces

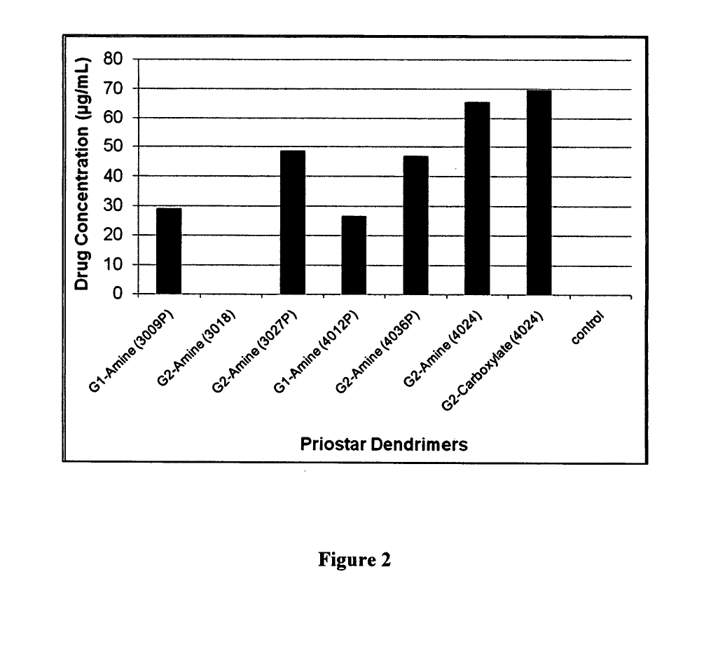

An excess of the cosmetic and nutrient Active Material, vitamin D3, (Sigma) was added to vials containing PEHAM dendritic polymers G=1.0 and G=2.0 with PETGE or TMPTGE cores and amine surfaces, partially PEGylated (25% PEG-550) amine surfaces, or carboxylate surfaces, dissolved in 20% (v/v) aqueous methanol at 1 wt % concentrations. These suspensions were briefly ultrasonicated in a water bath at RT, then incubated overnight at 32° C. and 100 rpm in a shaking water bath, and allowed to equilibrate at RT. The suspensions were filtered through a 0.2-μm Nylon syringe filter to remove not encapsulated Active Material. Solvent was removed and the residue redispersed in DI water. The samples were analyzed for dendrimer-associated vitamin D3 by HPLC. Association results are expressed in ng Active Material per mL solution. The results showed enhanced vitamin D3 solubility for all but one PEHAM dendritic polymer (see FIG. 2). FIG. 2 shows the solubility enhancement of the cosmetic and nutrient Active Material, vitamin D3 in the presence of PEHAM dendritic polymers G=1.0 and G=2.0 with PETGE (columns 4-7 from left) or TMPTGE (columns 1-3 from left) cores and amine surfaces, partially PEGylated (25% PEG-550) amine surfaces (denoted with letter “P”), or carboxylate surfaces. The pure Active Material as control remains below detection limit of 50 ng/mL.

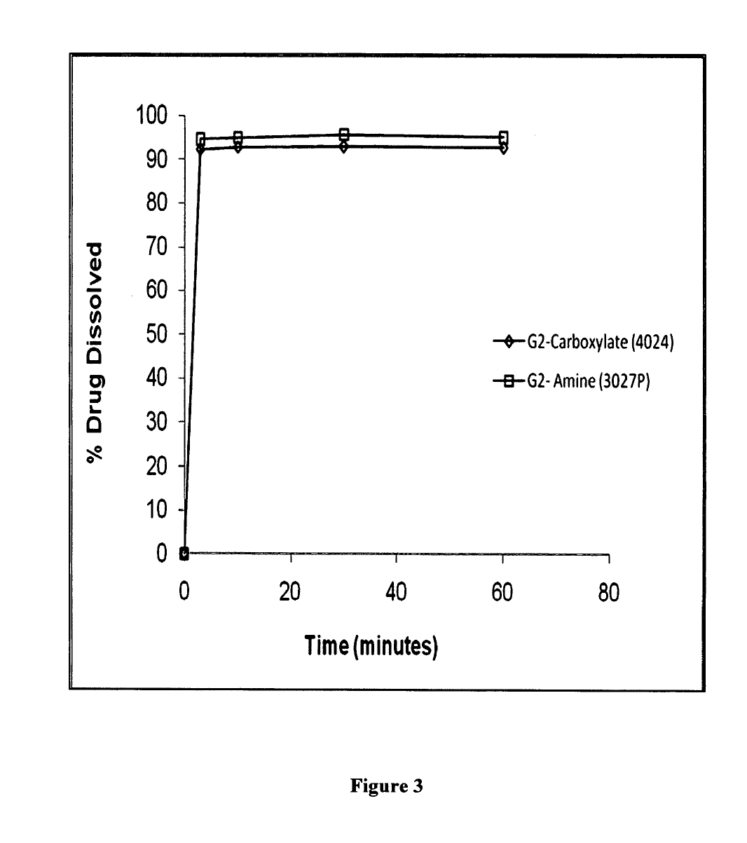

The effect of PEHAM dendritic polymers on the dissolution rate of vitamin D3 in PBS solution is shown in FIG. 3. This FIG. 3 shows the effect of PEHAM dendritic polymers on the dissolution rate of vitamin D3 in PBS solution. The otherwise poorly water soluble Active Material dissolves completely within 5 min. in the presence of PEHAM excipients.

### Example 5

PEHAM dendritic polymer G=1 with PETGE core and PIPZ surface as carrier in prodrug approach. The therapeutic Active Material, indomethacin, has been chemically bound to interior hydroxyl groups of a PEHAM dendritic polymer, creating a prodrug. Hydrolysis of the dendrimer-indomethacin complex and release of the unaltered drug disclose association of an Active Material through chemical bonding.
[(C)=PETGE; (IF1)=OH; (EX1)=PIPZ; (IF2)=OH; (BR1)=PETGE; (IF3)=OH; (EX2)=PIPZ; (TF)=Secondary NH; G=1.5]

### A. Protection of Terminal Piperazine NH Groups to Prevent Surface Attachment of Indomethacin

PEHAM dendritic polymer (50 mg, 0.016 mmol) and tri(ethyleneglycol)methylether p-nitrophenyl carbonate (250 mg, 0.064 mmol, 4 equiv.) were mixed in 3 mL of MeOH and stirred for 4 days. The reaction mixture was transferred into a dialysis bag (1,000 Dalton dialysis membrane, 18 mm diameter, 10 cm in length, Spectra/Por®, Spectrum Laboratories) and dialyzed in water. The purified product was isolated by lyophilization to give a yellow solid (41 mg, 36% yield). Its spectra are as follows:

¹H NMR (CDCl₃): δ 4.30-4.15 (18H, br), 4.00-3.80 (31H, br), 3.70-3.20 (267H, br), 2.75-2.20 (152H, br); and

¹³C NMR (125 MHz, CDCl₃): δ 156.2, 155.4, 152.4, 145.2, 125.4, 122.5, 73.4, 72.1, 70.8, 69.8, 66.8, 66.6, 66.5, 64.8, 61.0, 60.9, 59.3, 53.4, 45.8, 44.9, 44.3, 44.0; and MALDI-TOF: C₂₄₅H₄₆₈N₃₂O₁₀₀; Calc. 5459, found 5471 [M]+amu (broad signals).

B. Reaction of Surface Protected PEHAM Dendrimer with Indomethacin

The triethyleneglycol-protected PEHAM dendritic polymer (80.0 mg, 0.015 mmol) and indomethacin (95.0 mg, 0.27 mmol, 18 equiv.) were dissolved in 5 mL of methylene-chloride, then DCC (60.0 mg, 0.3 mmol, 20 equiv) was added under mechanical stirring. After 24 hours, the solvent was removed, the remaining solid residue suspended in a small amount of acetone, and the suspension separated by centrifugation. The yellow solution was decanted and the solvent removed by rotary evaporation. The yellow residue was dissolved in MeOH and DMF (9:1) and first dialyzed in MeOH containing 5% DMF to improve the solubility, followed by dialysis in neat MeOH (1,000 Dalton dialysis membrane, 18 mm diameter, 10 cm in length, Spectra/Por®, Spectrum Laboratories). Evaporation of the dialysis bag content gave the desired product as a yellow solid (98 mg, 86% yield). Its spectra are as follows:

¹H NMR (CDCl₃): δ 8.01, 7.67-7.63 (m), 7.48-7.44 (m), 7.00-6.95 (m), 6.83-6.79 (m), 6.66-6.62 (m), 5.20-5.12 (br), 4.30-4.15 (m), 4.10-3.10 (m), 2.75-2.10 (m).

### C. Hydrolysis of PEHAM Dendrimer-Indomethacin Prodrug

The PEHAM-indomethacin prodrug (98 mg, 0.013 mmol) was dissolved in 10 mL of MeOH and 0.5 mL concentrated HCl under mechanical stirring. After 3 hours, the reaction was quenched with aqueous sodium hydrogen carbonate and dialyzed in water (1,000 Dalton dialysis membrane, 38 mm diameter, 5 cm in length, Spectra/Por®, Spectrum Laboratories). The content of the dialysis bag was filtered and the solid residue dried in an air stream to give a yellow solid (17 mg, fraction A). The filtrate was concentrated by rotary evaporation, decanted and solid parts removed by centrifugation. The supernatant yellow solution was the dried by rotary evaporation to give a yellow solid (57 mg, fraction C). The insoluble product from the flask was dissolved in acetone and dried by rotary evaporation to give a yellow solid (21 mg, fraction B). Fractions A-C were analyzed by ¹H NMR spectroscopy and MALDI-TOF MS. The desired product, i.e., the PEHAM dendritic polymer without attached indomethacin, was identified in fraction C by the peak in MALDI-TOF MS at m/z 5464 [M]⁺ and by its ¹H NMR spectrum, which was virtually identical to that of the starting material. Weight of fraction C is consistent with recovery of 83% of the PEHAM dendritic polymer. Fraction A was identified by ¹H NMR spectroscopy as indomethacin. The weight of fraction A is consistent with recovery of 58% of indomethacin. Fraction B was identified by MALDI-TOF MS as a mixture of fractions A and C and their spectra are as follows:

Fraction A (Recovered Indomethacin):

¹H NMR (CDCl₃): δ 7.67-7.63 (m), 7.48-7.45 (m), 6.97-6.95 (m), 6.83-6.80 (m), 4.05-3.95 (m, impurity), 3.82, 3.70-3.60 (m), 2.38, 2.00-1.00 (impurity).

Fraction C (Recovered PEHAM Dendritic Polymer):

¹H NMR (CDCl₃): δ 4.25-4.18 (br), 4.00-3.20 (br), 2.70-2.20 (br); and

MALDI-TOF: C₂₄₅H₄₆₈N₃₂O₁₀₀; Calc. 5459, found 5464 [M]⁺ amu (broad signals).

The following Scheme 1 illustrates the prodrug formulation.

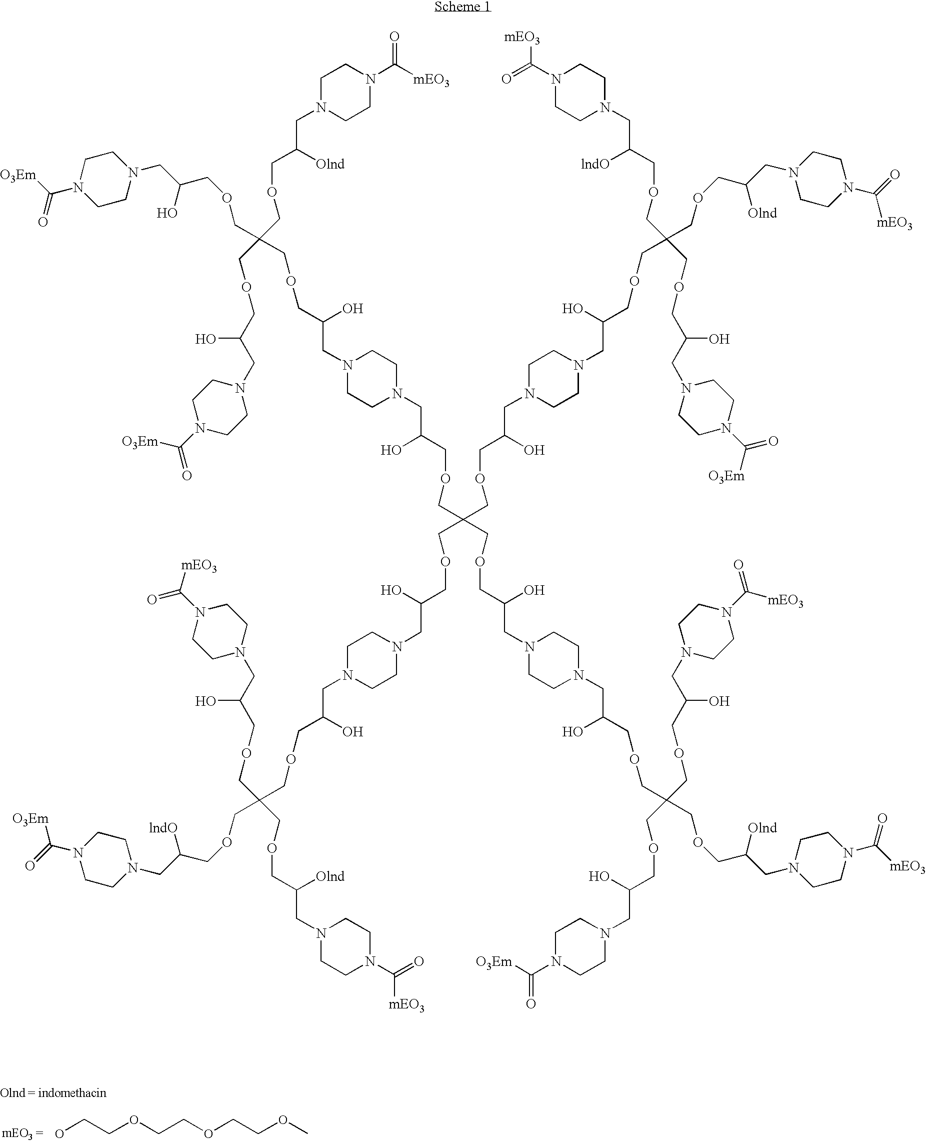

### Example 6

### Solubility Enhancement of an Active Material, Indomethacin, Associated with PEHAM Dendritic Polymer G=1.5 with PETGE Core and Poly(Ethylene Glycol), Molecular Weight 550, Surface in the Presence of PEG-E and Co-Excipient Sodium 4-Nitrophenylformiate and in the Absence of PEG

[(C)=PETGE; (IF1)=OH; (EX1)=PIPZ; (IF2)=OH; (BR1)=PETGE; (IF3)=OH; (EX2)=PIPZ; (EX3)=PEG-550; (TF)=Methoxy; G=1.5]

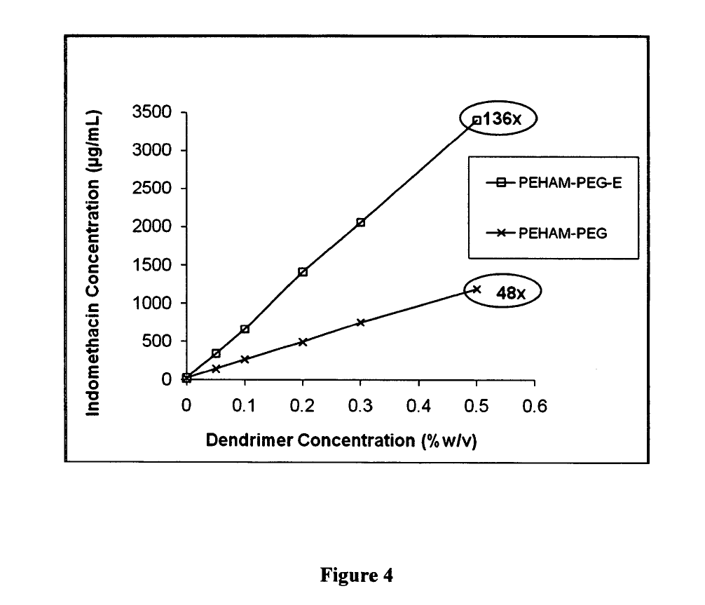

An excess of the drug indomethacin (Alfa Aesar) was added to vials containing PEHAM dendritic polymer G=1.5 with PETGE core and poly(ethylene glycol), molecular weight 550, (PEG) surface dissolved in DI water at concentrations of 0, 0.1, 0.2, 0.3, 0.4 and 0.5 percent (weight/volume). Sodium 4-nitrophenylformiate was added to one vial as a co-excipient in order to enhance the association of indomethacin with PEHAM. These suspensions were briefly ultrasonicated in a water bath at RT, then incubated overnight at 32° C. and 100 rpm in a shaking water bath, and allowed to equilibrate at RT. The suspensions were filtered through a 0.2-μm Nylon syringe filter to remove not encapsulated drug. Filtrates were analyzed for dendrimer-associated indomethacin by UV spectroscopy at 320 nm on a Perkin Elmer Lambda 2 UV/VIS Spectrometer. Association results are expressed as molar ratio indomethacin (I)/dendrimer (D). The results show enhanced indomethacin solubility for both PEHAM dendrimers with increasing PEHAM concentration; however, the increase is higher for PEHAM-PEG-E with co-excipient sodium 4-nitrophenylformiate (increase 136× at 0.5% w/v) compared to PEHAM-PEG without co-excipient (increase 48× at 0.5% w/v) (see FIG. 4). This FIG. 4 shows solubility enhancement of indomethacin in the presence of PEHAM dendritic polymer G=1.5 with PETGE core and poly(ethylene glycol), molecular weight 550, surface in the presence (PEG-E) and absence (PEG) of co-excipient sodium 4-nitrophenylformiate. The solubility enhancement factor x is shown as well.

### Example 7

### Comparison of loading efficiencies of an Active Material, Indomethacin, into PEHAM Dendritic Polymer G=1.0 with PETGE Core and PIPZ Surface and PAMAM Dendritic Polymer G=4 with EDA Core and Amine Surface

An excess of the Active Material, indomethacin (Alfa Aesar) was added to vials containing PEHAM dendritic polymer G=1.0 with PETGE core and piperazine (NH) surface and PAMAM dendritic polymer G=4.0 with EDA core and amine surface, each dissolved in DI water at concentrations of 0, 0.1, 0.2, 0.3 and 0.4 percent (w/v). These suspensions were briefly ultrasonicated in a water bath at RT, then incubated overnight at 32° C. and 100 rpm in a shaking water bath, and allowed to equilibrate at RT. The suspensions were filtered through a 0.2-μm Nylon syringe filter to remove not encapsulated drug. Filtrates were analyzed for dendrimer-associated indomethacin by UV spectroscopy at 320 nm on a Perkin Elmer Lambda 2 UV/VIS Spectrometer. Association results are expressed as μg indomethacin per mL solution. The results show enhanced indomethacin solubility for PEHAM dendritic polymer (52× compared to pure Active Material) compared to PAMAM dendritic polymer (28× compared to pure drug) at identical dendrimer concentrations despite the lower generation of the PEHAM dendritic polymer (see FIG. 5). This FIG. 5 shows the solubility enhancement of indomethacin in the presence of PEHAM dendritic polymers G=1.0 with PETGE core and piperazine (NH) surface and PAMAM dendritic polymer G=4.0 with EDA core and amine surface. The solubility enhancement factor x is shown as well.

### Example 8

### Altered Pharmacological Release Profiles of an Active Material, Indomethacin, Associated with PEHAM Dendritic Polymers G=1.5 with PETGE core And Piperazine (NH) or Poly(Ethylene Glycol), Molecular Weight 550, (PEG-E) surface, G=2.0 with PETGE Core and Hydroxyl (OH) Surface, and G=2.5 with TPEGE Core and Sodium Carboxylate (COONa) Surface

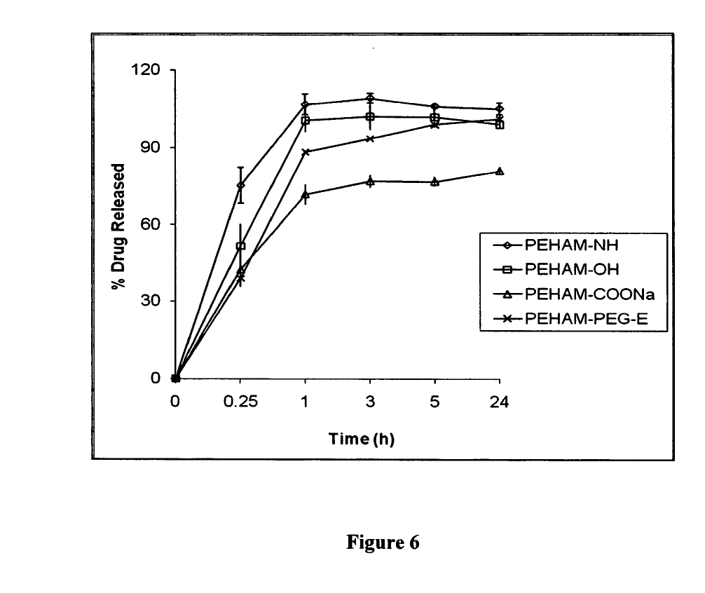

PEHAM dendritic polymer-indomethacin association complexes were analyzed for in vitro release by dialysis (Spectra/Por Membrane MWCO-1000; Fisher) against 20 mL phosphate buffered saline (PBS) at a pH of 7.25 and 37° C. with constant rocking. At scheduled time intervals of 0.25, 1, 3, 5, and 24 hours, the outer compartment of the dialysis apparatus was analyzed for indomethacin by UV spectroscopy. Drug release between 40 and 80% occurred for PEHAM G=1.5 and G=2.0 formulations, with complete indomethacin release after 24 hours. The release profile from PEHAM G=2.5 was slower, reaching approx. 85% indomethacin release after 24 hours (see FIG. 6). FIG. 6 shows the altered pharmacological release profiles of indomethacin associated with PEHAM dendritic polymers G=1.5 with PETGE core and piperazine (NH) or poly(ethylene glycol), molecular weight 550, (PEG-E) surface, G=2.0 with PETGE core and hydroxyl (OH) surface, and G=2.5 with TPEGE core and sodium carboxylate (COONa) surface in PBS solution at pH 7.25 and 37° C.

Although the invention has been described with reference to its preferred embodiments, those of ordinary skill in the art may, upon reading and understanding this disclosure, appreciate changes and modifications which may be made which do not depart from the scope and spirit of the invention as described above or claimed hereafter.

1. A compound comprising PEHAM dendritic polymers associated with Active Materials wherein the dendritic polymers function as excipients for the enhancement of water solubility of poorly water soluble (hydrophobic) Active Materials or enhancement of oil solubility of poorly oil soluble (hydrophilic) Active Materials.

2. The dendritic polymers of claim 1 wherein the Active Materials are associated with the dendritic polymers by one or more of the following: (a) by adsorption onto the surface, (b) encapsulation into the interior of the dendritic polymers, (c) a mixture of both (a) and (b) where these interactions are driven by one or more of the following: (i) electrostatic attraction, (ii) hydrogen bonding between dendritic polymers and Active Materials, or (iii) hydrophobic or hydrophilic interactions, or mixtures of these interactions.

3. The dendritic polymers of claim 1 wherein Active Materials are associated with dendritic polymers through chemical bonding to the surface or to internal functionalities (IF) of PEHAM dendritic polymers or both.

4. The dendritic polymers of claim 1 wherein Active Materials are associated with the dendritic polymers through chemical bonding to internal functionalities (IF) of the PEHAM dendritic polymers.

5. The dendritic polymers of claim 1 wherein the Active Material is cisplatin and the PEHAM dendritic polymer is G=2.5 with TPEGE core and sodium carboxylate (COONa) surface (TT).

6. The dendritic polymers of claim 1 wherein the Active Material is vitamin D3 and the PEHAM dendritic polymer is selected from G=1.0 and G=2.0 with PETGE or TMPTGE cores and amine, partially PEGylated amine, or carboxylate surfaces (TF).

7. The dendritic polymers of claim 1 wherein the PEHAM dendritic polymer is G=1.5 with PETGE core and poly(ethylene glycol), molecular weight 550, surface in the presence of PEG-E and co-excipient sodium 4-nitrophenylformiate.

8. The dendritic polymers of claim 3 wherein the bonding is done either directly between PEHAM dendritic polymers and Active Material molecules or via a linker that has a hydrolysable bond to the Active Material.

9. The dendritic polymer of claim 8 wherein the hydrolysable bond to the Active Material is labile by acid, base, enzyme, temperature, or light.

10. The dendritic polymers of claim 1 wherein an Active Material “Q” binds through appropriate chemical reaction mainly to the outside (TF) of the dendritic polymer and an Active Material “X” binds mainly to the interior (IF) of the dendritic polymer, thereby creating a combination of Active Materials.

11. The dendritic polymers of claim 10 wherein the Active Materials are: drugs providing a combination therapy or drug cocktail; or a drug and a diagnostic agent.

12. The dendritic polymers of claim 1 wherein a chemical entity with strong interaction to the Active Material and to the dendritic polymer is associated with the dendritic polymer through physical means prior to adsorption or encapsulation of the Active Material or together with the Active Material.

13. The dendritic polymers of claim 1 wherein a chemical entity with strong interaction to the Active Material and to the dendritic polymer is chemically attached to (IF) or (TF) prior to the Active Material's adsorption or encapsulation.

14. The dendritic polymers of claim 12 or 13 wherein the chemical entity acts as a co-excipient or co-encapsulant and enhances the Active Material's adsorption or encapsulation efficiency.

15. The dendritic polymers of claim 12 or 13 wherein the chemical entity is piperazine, 4-nitrophenylformiate or a derivative thereof.

16. The dendritic polymers of any one of claims 1 to 13 wherein loading efficiency of the Active Material into said dendritic polymers is higher than those observed for other dendrimers and is achieved at a lower dendrimer generation.

17. The dendritic polymers of any one of claims 1 to 13 wherein the high thermal stability of said dendritic polymers allows thermal sterilization of formulations containing the Active Material.

18. A formulation comprising the dendritic polymers of any one of claims 1 to 13 with Active Materials and having at least one additional pharmaceutically-acceptable, cosmetically-acceptable or nutrient-acceptable excipient, diluent, carrier, surfactant, desiccant, or solvent.

19. The formulation of claim 18 that is stored, provided as a powder mixture, and re-dissolved retaining its activity prior to application.

20. The formulation of claim 18 as a solid mixture and pressed into tablets.

21. The formulation of claim 18 prepared by concentration of mixed solutions, stored, and provided as a suspension or paste filled into a capsule.

22. The formulation of claim 18 which is administered by an oral route, ampoule, intravenous injection, intramuscular injection, transdermal application, intranasal application, intraperitoneal administration, subcutaneous injection, ocular application, as wipes, sprays, gauze or other means for use at a surgical incision, near scar formation sites, or site of a tumor growth or removal or near or within a tumor.

23. The formulation of claim 18 wherein the Active Material is a drug and the formulation provides a more desirable pharmacological profile.

24. The formulation of claim 18 wherein the Active Material is a cosmetic or nutrient and the formulation improves the interaction with the body and desired performance.

25. A process for preparing the dendritic polymers associated with Active Materials of claims 1-4, 12 or 13 by: (a) mixing a solid Active Material with pre-dissolved PEHAM dendritic polymer, (b) solid PEHAM dendritic polymer is mixed with pre-dissolved Active Material, (c) both Active Material and dendrimer are mixed as solids and then dissolved at the same time, or (d) both Active Material and PEHAM dendritic polymer are pre-dissolved and then mixed, wherein in the final mixture prepared by (a), (b) or (c) the dendritic polymer is more strongly associated with the Active Material than the bulk solution is associated with the Active Material so that there is a driving force for the Active Material to interact with the dendritic polymer.
